SEQUENCE LISTING (1 sequence)

SEQ ID NO: 1 - DNA, 729 bp, Homo Sapiens
cttgatggcatcgcggggaagggactaagtccagataatgtcctctgaggcttcggccccgtgggcaggacacacctcctgcgggcctattcaataatcagttaaatcacctgaagcacacgcatttccggggaccgctccgggcatcctggcttgagggtagagtgggcggaggttcctaagggagaggtggggctcgggctgaatccctcgttggggggcatctgggtcaagtggcttccctggcagcacagtcacggggaggccctctctcattgggcagaagctaagtccgaagccgcgcccctcctgggaggttgaactgtggtgcaggaaagcctcaagtagaggagggttgaggcttcaatccagcactttgctcgggtcacggcctcctcctggctcccaggaccccaccataggcagaggcaggccttcctacaccctactccctgtgcctccaggctcgactagtccctagcactcgacgactgagtctctgaggtcacttcaccgtggtctccgcctcacccttggcgctggaccagtgagaggagagggctggggcgctccgctgagccactcctgcgcccccctggccttgtctacctcttgcccccgaagggttagtgtcgagctcaccccagcatcctacaacctcctggtggccttgccgcccccacaaccccgaggtataaagccaggtacaccaggcaggggacgcaccaa

(19) United States
(12) Patent Application Publication
(10) Pub. No.: US 2003/0213006 A1
(43) Pub. Date: Nov. 13, 2003

(54) Beta-hcg promoter based tumor restrictive gene expression for cancer theraphy

(76) Inventors: Ri-Hyun Back, Kobe (JP); Thomas A. Gardner, Zionsville, IN (US); Akinobu Gotoli, Higashimada-ku (JP)

Correspondence Address: PENNIE AND EDMONDS, 1155 AVENUE OF THE AMERICAS, NEW YORK, NY, 100362711

(21) Appl. No.: 10/296,667

(22) PCT Filed: May 24, 2001
(86) PCT No.: PCT/US01/16942

(51) Int. Cl. A01K 67/00; C07H 21/04; C12P 21/02; C12N 5/06; C07K 14/47; A61K 31/522; A61K 48/00; A61K 31/513

(52) U.S. Cl. 800/8; 536/23.2; 424/93.2; 514/44; 530/350; 435/69.1; 435/320.1; 435/325; 514/263.32; 514/269

(57) ABSTRACT

The present invention relates to promoters, enhancers and other regulatory elements that direct expression within tumor cells, comprising nucleotide sequences from the 5′ regulatory region, and transcriptionally active fragments thereof, that control expression of a testicular carcinoma related protein, β-HCG. Specifically provided are expression vectors, host cells and transgenic animals wherein an β-HCG regulatory region is capable of controlling expression of a heterologous gene, over-expressing an endogenous gene or an inhibitor of a pathological process or knocking out expression of a specific gene believed to be important in cancer development and/or progression. The invention also relates to methods for using said vectors, cells and animals for screening candidate molecules for agonists and antagonists of cancer development and/or progression. The invention further relates to compositions and methods for modulating expression of compounds within tumor cells, and to screening compounds that modulate expression within tumor cells. Methods for using the molecules and compounds identified by the screening assays for therapeutic treatments also are provided.

### 1. INTRODUCTION

The present invention relates to promoters, enhancers and other regulatory elements that control the expression of a testicular carcinoma related protein, human chorionic gonadotropin beta-subunit (“β-HCG”). In particular, it relates to compositions comprising nucleotide sequences from the 5′ regulatory region, and transcriptionally active fragments thereof, that control expression of β-HCG. Specifically provided are expression vectors, host cells and transgenic animals wherein the β-HCG promoter is capable of controlling expression of a heterologous gene, over-expressing an endogenous gene or an inhibitor of a pathological process or knocking out expression of a specific gene believed to be important in cancer development and/or progression. The invention also relates to methods for using said vectors, cells and animals for screening candidate molecules for agonists and antagonists of cancer development and/or progression.

The present invention also relates to compositions and methods for modulating expression of compounds that are involved in cancer development and/or progression. The invention further relates to screening compounds that modulate expression during cancer development and/or progression. Methods for using molecules and compounds identified by the screening assays for therapeutic treatments also are provided.

The present invention further relates to methods and compositions comprising the β-HCG promoter, and transcriptionally active fragments thereof, which are capable of selectively driving expression of a heterologous gene in a tissue specific manner. More particularly, the promoters are capable of selectively increasing expression of heterologous genes within various tumors. Thus, due to the tissue specificity of the promoters of the invention, therapeutic gene delivery can be targeted to cancer cells, while sparing delivery of the therapeutic genes to normal, nonneoplastic cells. Moreover, the tissue specific expression provides the added advantage of allowing for administration of a therapeutic gene not only via direct application, such as by injection, but also systemically to the body via intravenous administration, oral administration or the like, because gene expression will be limited and localized to specific cell types.

### 2. BACKGROUND OF THE INVENTION

### 2.1 Gene Therapy

Somatic cell gene therapy is a strategy in which a nucleic acid, typically in the form of DNA, is administered to alter the genetic repertoire of target cells for therapeutic purposes. Although research in experimental gene therapy is a relatively young field, major advances have been made during the last decade. (Arai, Y., et al., 1997, Orthopaedic Research Society, 22:341). The potential of somatic cell gene therapy to treat human diseases has caught the imagination of numerous scientists, mainly because of two recent technologic advancements. Firstly, there are now numerous viral and non-viral gene therapy vectors that can efficiently transfer and express genes in experimental animals in vivo. Secondly, increasing support for the human genome project will allow for the identity and sequence of the estimated 80,000 genes comprising the human genome in the very near future.

Gene therapy was originally conceived of as a specific gene replacement therapy for correction of heritable defects to deliver functionally active therapeutic genes into targeted cells. Initial efforts toward somatic gene therapy relied on indirect means of introducing genes into tissues, called ex vivo gene therapy, e.g., target cells are removed from the body, transfected or infected with vectors carrying recombinant genes and re-implanted into the body (“autologous cell transfer”). A variety of transfection techniques are currently available and used to transfer DNA in vitro into cells; including calcium phosphate-DNA precipitation, DEAE-Dextran transfection, electroporation, liposome mediated DNA transfer or transduction with recombinant viral vectors. Such ex vivo treatment protocols have been proposed to transfer DNA into a variety of different cell types including epithelial cells (U.S. Pat. No. 4,868,116; Morgan and Mulligan WO87/00201; Morgan et al., 1987, Science 237:1476-1479; Morgan and Mulligan, U.S. Pat. No. 4,980,286), endothelial cells (WO89/05345), hepatocytes (WO89/07136; Wolff et al., 1987, Proc. Natl. Acad. Sci. USA 84:3344-3348; Ledley et al., 1987 Proc. Natl. Acad. Sci. 84:5335-5339; Wilson and Mulligan, WO89/07136; Wilson et al., 1990, Proc. Natl. Acad. Sci. 87:8437-8441), fibroblasts (Palmer et al., 1987, Proc. Natl. Acad. Sci. USA 84:1055-1059; Anson et al., 1987, Mol. Biol. Med. 4:11-20; Rosenberg et al., 1988, Science 242:1575-1578; Naughton & Naughton, U.S. Pat. No. 4,963,489), lymphocytes (Anderson et al., U.S. Pat. No. 5,399,346; Blaese, R. M. et al., 1995, Science 270:475-480) and hematopoietic stem cells (Lim, B. et al. 1989, Proc. Natl. Acad. Sci. USA 86:8892-8896; Anderson et al., U.S. Pat. No. 5,399,346).

Direct in vivo gene transfer recently has been attempted with formulations of DNA trapped in liposomes (Ledley et al., 1987, J. Pediatrics 110:1), in proteoliposomes that contain viral envelope receptor proteins (Nicolau et al., 1983, Proc. Natl. Acad. Sci. U.S.A. 80:1068) and DNA coupled to a polylysine-glycoprotein carrier complex. In addition, “gene guns” have been used for gene delivery into cells (Australian Patent No. 9068389). It even has been speculated that naked DNA, or DNA associated with liposomes, can be formulated in liquid carrier solutions for injection into interstitial spaces for transfer of DNA into cells (Felgner, WO90/11092).

Numerous clinical trials utilizing gene therapy techniques are underway for such diverse diseases as cystic fibrosis and cancer. The promise of this therapeutic approach for dramatically improving the practice of medicine has been supported widely, although there still are many hurdles that need to be passed before this technology can be used successfully in the clinical setting.

Perhaps, one of the greatest problems associated with currently devised gene therapies, whether ex vivo or in vivo, is the inability to control expression of a target gene and to limit expression of the target gene to the cell type or types needed to achieve a beneficial therapeutic effect.

The concept of delivery and expression of therapeutic toxic genes to tumor cells through the use of tissue-specific promoters has been well recognized. This approach decreases the toxic effect of therapeutic genes on neighboring normal cells when vector (virus, liposome, etc.) gene delivery results in the infection of the normal cells as well as the cancerous cells. Examples include the uses of α-fetoprotein promoter to target hepatoma cells (Koryama, et al., 1991, Cell Struct. Punct., 16:503-510), the carcinoembryonic antigen (CEA) promoter for gastric carcinoma (Tanaka, et al., 1996, Cancer Research, 46: 1341-1345), the tyrosinase promoter to kill melanoma cells (Vile, et al., 1994, Cancer Research, 54:6228-6234), the bone morphogenic protein promoter for brain to target glioma cells (Shimizu, K., 1994, Nipson Rinsbo, 52:3053-3058), and the osteocalcin promoter to kill osteocarcinoma and prostate cancers (Ko, S. et al., 1996, Cancer Research, 56: 4614-4619; Gardener, et al., 1998, Gene Therapy and Molecular Biology, 2:41-58). Molecular therapeutic strategies such as gene therapy through use of tissue and tumor-restricted promoters are being used with increasing frequency. The key components of a gene therapy approach include: i) the selection of appropriate tissue-specific or tumor-restricted promoters, which, in some instances, may be inducible by a hormone, vitamin, an antibiotic, drug or heavy metal; ii) the selection of therapeutic (or toxic) genes; iii) the appropriate vectors, such as retrovirus, adenovirus, liposomes, etc. Key to targeting the appropriate tumor tissue while sparing the normal host tissue is a promoter that can home the therapeutic genes to only those tissues which use the chosen promoter.

### 2.2 Expression of the β-HCG Protein

Human chorionic gonadotropin beta-subunit (β-HCG) and alpha-fetoprotein have been used as diagnostic markers of testicular cancer. The elevation of these tumor markers in radioimmunoassays is more sensitive than radiologic evaluations of metastatic disease. The elevation of β-HCG serum levels is found primarily in non-seminomatous germ cell tumors, but the production of this protein also occurs in both primary and recurrent metastatic seminomas. Immunohistochemical studies have revealed further that the β-HCG protein is produced by, e.g., testicular cancers at both primary and metastatic sites.

Although great strides have been made in the therapeutic approach to testicular carcinomas, it remains the number one malignancy in men 20-40 years of age. Prior to 1970, the disease was associated with approximately 80% mortality. Over the last twenty years, the development of effective chemotherapeutics combined with an aggressive surgical approach to this disorder has had an extremely high success rate. Currently, the mortality associated with testicular cancer has dropped to less than 10%. Unfortunately, the morbidity and the mortality associated with this cancer and the treatments are still having a significant impact on the quality of life for men suffering from testicular cancers. The development of novel molecular therapeutics that can be combined with chemotherapeutic and/or surgical approaches may allow the maximizing of therapeutic benefits while minimizing potential therapy-related toxicities.

Thus, in view of the current deficiencies with regards to treating various cancers, including, but not limited to, testicular cancers, novel therapeutic treatments for these cancers are urgently needed. The present invention meets these needs and provides a useful model for modulating, diagnosing and/or treating such cancers.

### 3. SUMMARY OF THE INVENTION

The invention disclosed herein provides a model for tumor-specific gene transcription. The invention is based in part on the functional characterization described herein of a the novel β-HCG promoter, which is a promoter found to be active only in various cancers, including, but not limited to, testicular cancers.

The present invention provides compositions and methods for screening compounds that modulate expression of compounds during cancer development and/or progression. In particular, it provides compositions comprising nucleotides from the β-HCG 5′ regulatory region, and transcriptionally active fragments thereof, as well as nucleic acids that hybridize under highly stringent and moderately stringent conditions to such nucleotides, that control the expression of the cancer-related protein, β-HCG. Specifically provided are expression vectors comprising the β-HCG 5′ regulatory region, and transcriptionally active fragments thereof, operably associated to a heterologous reporter gene, and host cells and transgenic animals containing such vectors. The invention also provides methods for using such vectors, cells and animals for screening candidate molecules for agonists and antagonists of various cancers. Methods for using molecules and compounds identified by the screening assays for therapeutic treatments also are provided.

For example, and not by way of limitation, a composition comprising a reporter gene is operatively linked to a tumor-specific regulatory sequence, herein called the β-HCG regulatory region. The β-HCG driven reporter gene is expressed as a transgene in animals. The transgenic animal, and cells derived from cancerous cells within the transgenic animal, can be used to screen compounds for candidates useful for modulating various cancers. Without being bound by any particular theory, such molecules are likely to interfere with the function of trans-acting factors, such as transcription factors, as well as cis-acting elements, such as promoters and enhancers involved in various cancers. As such, they are potentially powerful candidates for treatment of such cancers, including, but not limited to, primary, metastatic and recurrent testicular carcinomas.

In one embodiment, the invention provides methods for high throughput screening of compounds that modulate specific expression of genes within tumor cells. In this aspect of the invention, cells from cancerous tissues are removed from the transgenic animal and cultured in vitro. The expression of the reporter gene is used to monitor tumor-specific gene activity. In a specific embodiment, green fluorescent protein (GFP) is the reporter gene. In another embodiment, firefly luciferase is the reporter gene. Compounds identified by this method can be tested further for their effect on non-cancerous cells in normal animals.

In another embodiment, the transgenic animal model of the invention can be used for in vivo screening to test the mechanism of action of candidate drugs for their effect on cancerous cells. Specifically, the effects of the drugs on various cancers including, but not limited to, primary, metastatic and recurrent testicular carcinomas, can be assayed.

In another embodiment, a gene therapy method for treating cancer development and/or progression is provided. β-HCG regulatory sequences are used to drive tumor-specific expression of drugs or toxins and introduced into various cancer cells. The method comprises introducing an β-HCG regulatory sequence operatively associated with a drug or toxin gene into a cancer cell.

The invention further provides methods for screening for novel transcription factors that modulate the β-HCG regulatory sequence. Such novel transcription factors identified by this method can be used as targets for treating various cancers.

### 4. BRIEF DESCRIPTION OF THE FIGURES

FIG. 1 illustrates the 729 bp β-HCG promoter. The start site of the promoter is at −370 relative to the start of transcription. Otani et al., 1988, J. Biol. Chem., 263:7322-7329. The 729 base pair fragment was TA-cloned and cut out of the TOPO vector at Hind III and Xho I and inserted into PGL3.

FIGS. 2A-2C illustrate the construction of β-HCG-TK (FIG. 2A), β-HCG-CD (FIG. 2B), and β-HCG-Ela (FIG. 2C) recombinant adenoviral shuttle vector. These vectors have been used to construct recombinant replication defective (Ad-β-HCG-TK and Ad-β-HCG-CD) and replication restrictive (Ad-β-HCG-Ela) adenoviral vectors.

FIG. 3 demonstrates the relative luciferase activity of β-HCG in testicular cancer cell lines (JAR, NEC-8, NEC-14) and non-testicular cell lines (PC-3, WH, DU-145). Each of the non-testicular cell lines demonstrate minimal luciferase activity relative to the pGL3 plasmid. On the other hand, the testicular cancer cell lines express 500 to 2000 fold more luciferase activity under the regulation of the β-HCG promoter.

FIGS. 4A-4B demonstrate the ability of the Ad-β-HCG-TK virus to significantly inhibit growth of testicular cancer cell line NEC-8 (FIG. 4A) while demonstrating minimal cytotoxic effect on the prostate cancer cell line PC-3 (FIG. 4B).

### 5. DETAILED DESCRIPTION OF THE INVENTION

The present invention provides promoters, enhancers and other regulatory elements that direct expression within tumors, comprising nucleotide sequences from the 5′ regulatory region, and transcriptionally active fragments thereof, that control expression of a β-HCG protein. Specifically provided are expression vectors, host cells and transgenic animals wherein an β-HCG regulatory region is capable of controlling expression of a heterologous gene, over-expressing an endogenous tumor gene or an inhibitor of a pathological process or knocking out expression of a specific gene believed to be important in cancer development and/or progression. Examples of such cancers include, but are not limited to, primary, metastatic and recurrent testicular carcinomas.

The invention also provides methods for using said vectors, cells and animals for screening candidate molecules for agonists and antagonists of cancer development and/or progression. In an alternative embodiment, the invention provides compositions and methods for modulating expression of compounds that are involved in cancer development and/or progression, and to screening compounds that modulate expression during cancer development and/or progression. Methods for using the molecules and compounds identified by the screening assays for therapeutic treatments also are provided.

The invention further provides methods of treating and/or ameliorating cancers and other diseases and disorders, including, but not limited to, primary, metastatic and recurrent testicular carcinomas.

The invention is based, in part, on the discovery that nucleotide sequences encoding toxic and/or therapeutic coding sequences contained within vectors (i.e. viral vectors) can be administered in a cell and tissue specific manner, with the use of promoters which allow for tissue specific expression of the nucleotide sequences. Further, because the vectors of the invention utilize these promoters to control the expression of toxic and/or therapeutic coding sequences, the vectors of the invention are effective therapeutic agents not only when administered via direct application, but also when administered systemically to the body, because the toxic and/or therapeutic coding sequences will be expressed only in specifically targeted cells, i.e., within cells that express β-HCG.

Taking advantage of this feature, the methods of the present invention are designed to efficiently transfer one or more DNA molecules encoding therapeutic agents to a site where the therapeutic agent is necessary. The methods involve the administration of a vector containing DNA encoding translational products (i.e. therapeutic proteins) or transcriptional products (i.e. antisense or ribozymes) within a mammalian host to a site where the translational product is necessary. Once the vector infects cells where the therapeutic agent is necessary, the coding sequence of interest, i.e., thymidine kinase, is expressed, thereby amplifying the amount of the toxic and/or therapeutic agent, protein or RNA.

The present invention relates also to pharmaceutical compositions comprising vectors containing DNA for use in treating and/or ameliorating cancers and other diseases and disorders, including, but not limited to, primary, metastatic and recurrent testicular carcinomas. The compositions of the invention generally are comprised of a bio-compatible material containing the vector containing DNA encoding a therapeutic protein of interest, i.e., thymidine kinase, growth factors, etc. A bio-compatible composition is one that is in a form that does not produce an allergic, adverse or other untoward reaction when administered to a mammalian host.

The invention overcomes shortcomings specifically associated with current recombinant protein therapies for treating and/or cancers and other diseases and disorders, including, but not limited to, primary, metastatic and recurrent testicular carcinomas. First, direct gene transfer is a rational strategy that allows transfected cells to (a) make physiological amounts of therapeutic protein, modified in a tissue- and context-specific manner, and (b) deliver this protein to the appropriate cell surface signaling receptor under the appropriate circumstances. Exogenous delivery of such molecules is expected to be associated with significant dosing and delivery problems. Second, repeated administration, while possible, is not required with the methods of the invention because various promoters, including inducible promoters, can be used to control the level of expression of the therapeutic protein of interest. Further, integration of transfected DNA can be associated with long term recombinant protein expression.

Described in detail below, in Sections 5.1 and 5.2, are nucleotide sequences of the β-HCG regulatory region, and expression vectors, host cells and transgenic animals wherein the expression of a heterologous gene is controlled by the β-HCG regulatory region. In Section 5.3, methods for using such polynucleotides (i.e., regulatory regions of the β-HCG gene) and fusion protein products, for screening compounds that interact with the regulatory region of the β-HCG gene are described. This section describes both in vivo and in vitro assays to screen small molecules, compounds, recombinant proteins, peptides, nucleic acids, antibodies, etc. which bind to or modulate the activity of the β-HCG regulatory region. Section 5.4 describes methods for the use of identified agonists and antagonists for drug delivery or gene therapy. Finally, in Section 5.5, pharmaceutical compositions are described for using such agonists and antagonists to modulate cancer cell-related disorders. Methods and compositions are provided for treating various cancers including, but not limited to, primary, metastatic and recurrent testicular carcinomas.

### 5.1 Polynucleotides and Nucleic Acids of the Invention

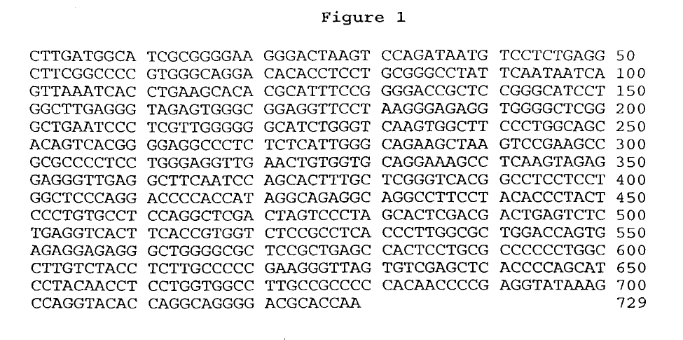

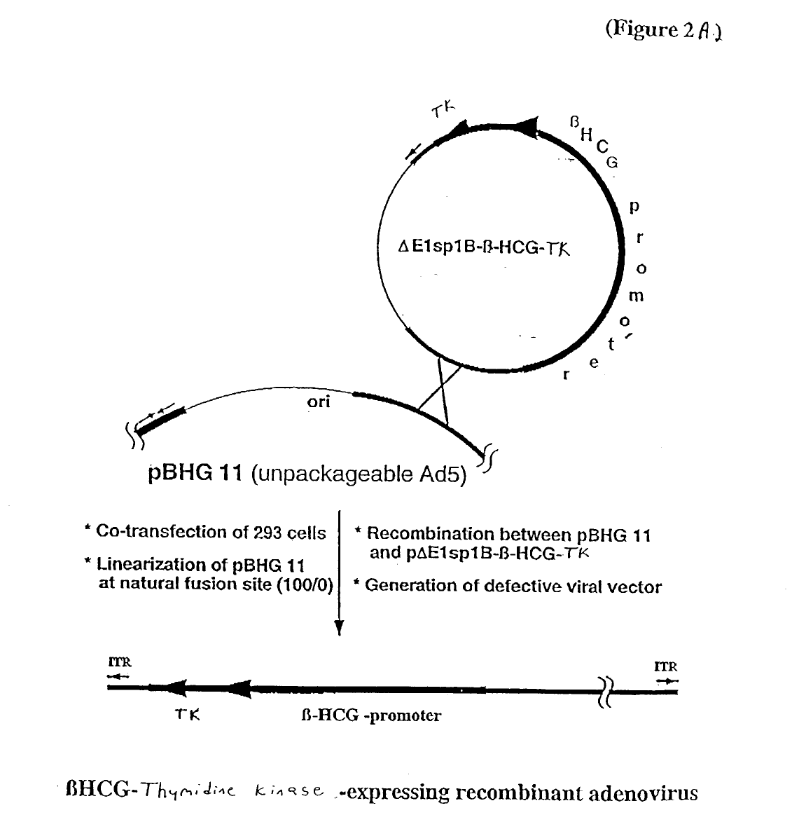

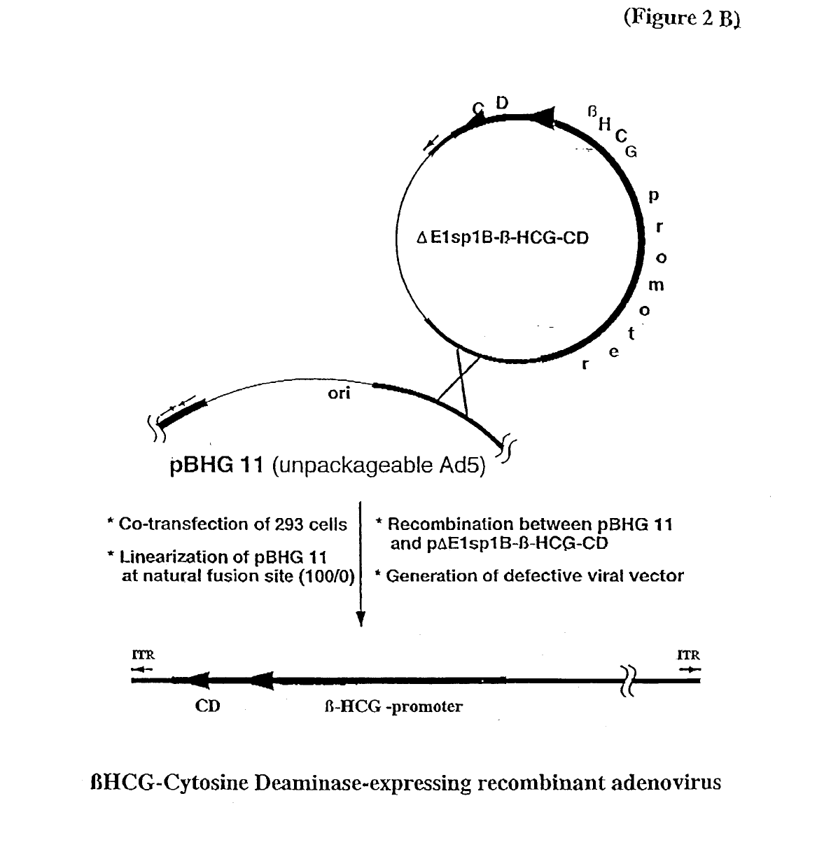

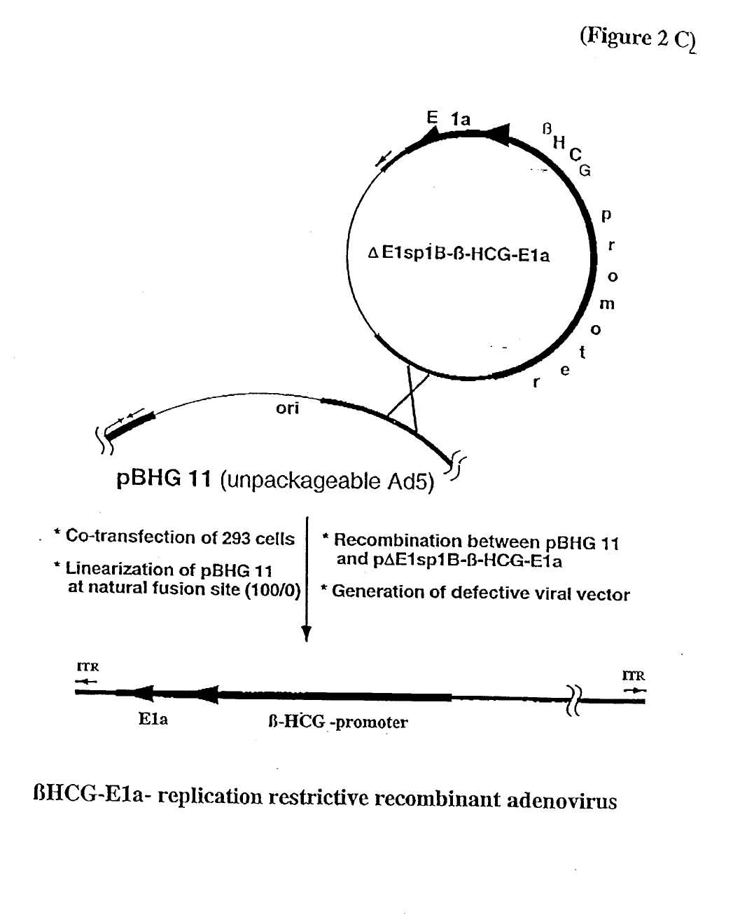

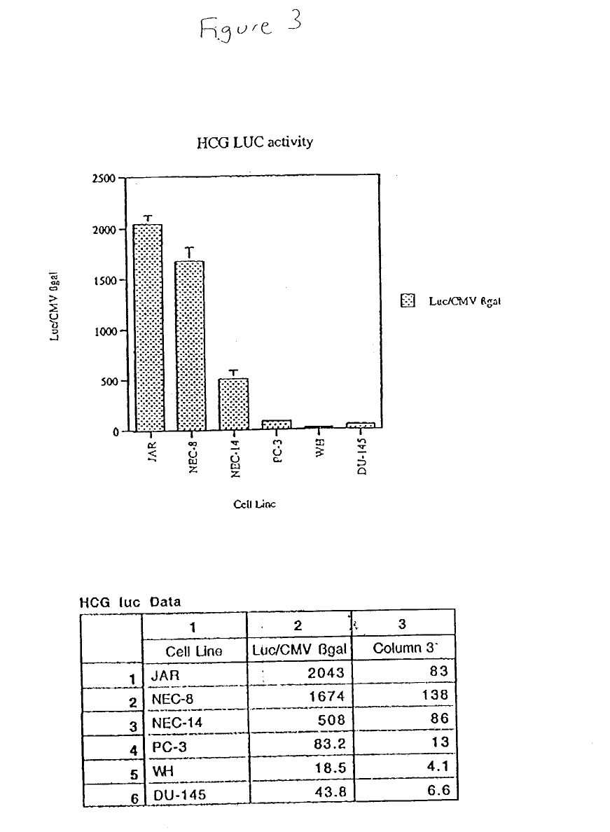

The present invention encompasses polynucleotide sequences comprising the 5′ regulatory region, and transcriptionally active fragments thereof, of the β-HCG gene. In particular, the present invention relates to a polynucleotide comprising the sequence, shown in FIG. 1, that is located immediately 5′ to the transcription start site of the β-HCG gene. In various embodiments, the polynucleotide may be 5000, 4000, 3000, 2000, 1000, preferably approximately 500 and more preferably 250 bp in length.

The invention further provides probes, primers and fragments of the β-HCG regulatory region. In one embodiment, purified nucleic acids consisting of at least 8 nucleotides (i.e., a hybridizable portion) of an β-HCG gene sequence are provided; in other embodiments, the nucleic acids consist of at least 20 (contiguous) nucleotides, 25 nucleotides, 50 nucleotides, 100 nucleotides, 200 nucleotides, 500, 1000, 2000, 3000, 4000 or 5000 nucleotides of a β-HCG sequence. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

In another embodiment, the nucleic acids are smaller than 20, 25, 35, 200 or 500 nucleotides in length. Nucleic acids can be single or double stranded. The invention also encompasses nucleic acids hybridizable to or complementary to the foregoing sequences. In specific aspects, nucleic acids are provided which comprise a sequence complementary to at least 10, 20, 25, 50, 100, 200, 500 nucleotides or the entire regulatory region of a β-HCG gene.

The probes, primers and fragments of the β-HCG regulatory region provided by the present invention can be used by the research community for various purposes. They can be used as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; and as a probe to “subtract-out” known sequences in the process of discovering other novel polynucleotides. Methods for performing the uses listed above are well known to those skilled in the art. References disclosing such methods include, without limitation, “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

The nucleotide sequences of the invention also include nucleotide sequences that have at least 65%, 70%, 75%, 80%, 85%, 90%, 95%, 98% or more nucleotide sequence identity to the nucleotide sequence depicted in FIG. 1, and/or transcriptionally active fragments thereof, which are capable of driving expression specifically within cancers, including, but not limited to, primary, metastatic and recurrent testicular carcinomas.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12 and a gap penalty of 4 can be used. In an alternate embodiment, alignments can be obtained using the NA_MULTIPLE_ALIGNMENT 1.0 program, using a GapWeight of 5 and a GapLengthWeight of 1.

The percent identity between two sequences can be determined using techniques similar to those described above, with or without allowing gaps. In calculating percent identity, typically only exact matches are counted.

The invention also encompasses:

(a) DNA vectors that contain any of the foregoing β-HCG regulatory sequences and/or their complements (i.e., antisense);

(b) DNA expression vectors that contain any of the foregoing β-HCG regulatory element sequences operatively associated with a heterologous gene, such as a reporter gene; and

(c) genetically engineered host cells that contain any of the foregoing β-HCG regulatory element sequences operatively associated with a heterologous gene such that the β-HCG regulatory element directs the expression of the heterologous gene in the host cell.

Also encompassed within the scope of the invention are various transcriptionally active fragments of this regulatory region. A “transcriptionally active” or “transcriptionally functional” fragment of the sequence depicted in FIG. 1 according to the present invention refers to a polynucleotide comprising a fragment of said polynucleotide which is functional as a regulatory region for expressing a recombinant polypeptide or a recombinant polynucleotide in a recombinant cell host. For the purpose of the invention, a nucleic acid or polynucleotide is “transcriptionally active” as a regulatory region for expressing a recombinant polypeptide or a recombinant polynucleotide if said regulatory polynucleotide contains nucleotide sequences which contain transcriptional information, and such sequences are operably associated to nucleotide sequences which encode the desired polypeptide or the desired polynucleotide.

In particular, the transcriptionally active fragments of the β-HCG regulatory region of the present invention encompass those fragments that are of sufficient length to promote transcription of a reporter gene when operatively linked to the β-HCG regulatory sequence and transfected into an β-HCG-expressing cell line. Typically, the regulatory region is placed immediately 5′ to, and is operatively associated with the coding sequence. As used herein, the term “operatively associated” refers to the placement of the regulatory sequence immediately 5′ (upstream) of the reporter gene, such that trans-acting factors required for initiation of transcription, such as transcription factors, polymerase subunits and accessory proteins, can assemble at this region to allow RNA polymerase dependent transcription initiation of the reporter gene.

In one embodiment, the polynucleotide sequence chosen may further comprise other nucleotide sequences, either from the β-HCG gene, or from a heterologous gene. In another embodiment, multiple copies of a promoter sequence, or a fragment thereof, may be linked to each other. For example, the promoter sequence, or a fragment thereof, may be linked to another copy of the promoter sequence, or another fragment thereof, in a head to tail, head to head, or tail to tail orientation. In another embodiment, a tumor-specific enhancer may be operatively linked to the β-HCG regulatory sequence, or fragment thereof, and used to enhance transcription from the construct containing the β-HCG regulatory sequence.

Also encompassed within the scope of the invention are modifications of this nucleotide sequence without substantially affecting its transcriptional activities. Such modifications include additions, deletions and substitutions. In addition, any nucleotide sequence that selectively hybridizes to the complement of the sequence depicted in FIG. 1 under stringent conditions, and is capable of activating the expression of a coding sequence is encompassed by the invention. Exemplary moderately stringent hybridization conditions are as follows: prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stririgency are as follows: e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3). Other conditions of high stringency which may be used are well known in the art. In general, for probes between 14 and 70 nucleotides in length the melting temperature (TM) is calculated using the formula: Tm(° C.)=81.5+16.6(log[monovalent cations (molar)])+0.41(% G+C)−(500/N) where N is the length of the probe. If the hybridization is carried out in a solution containing formamide, the melting temperature is calculated using the equation Tm(° C.)=81.5+16.6(log[monovalent cations (molar)])+0.41(% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

The β-HCG regulatory region, or transcriptionally functional fragments thereof, is preferably derived from a mammalian organism. Screening procedures which rely on nucleic acid hybridization make it possible to isolate any gene sequence from other organisms. The isolated polynucleotide sequence disclosed herein, or fragments thereof, may be labeled and used to screen a cDNA library constructed from mRNA obtained from appropriate cells or tissues (e.g., cancerous tissue) derived from the organism of interest. The hybridization conditions used should be of a lower stringency when the cDNA library is derived from an organism different from the type of organism from which the labeled sequence was derived. Low stringency conditions are well know to those of skill in the art, and will vary depending on the specific organisms from which the library and the labeled sequence are derived. For guidance regarding such conditions see, for example, Sambrook et al., 1989, Molecular Cloning, A Laboratory Manual, Second Edition, Cold Spring Harbor Press, N.Y., and Ausabel et al., 1989, Current Protocols in Molecular Biology, Green Publishing Associates and Wiley Interscience, N.Y., each of which is incorporated herein by reference in its entirety. Further, other mammalian β-HCG regulatory region homologues may be isolated from, for example, bovine or other non-human nucleic acid, by performing polymerase chain reaction (PCR) amplification using two primer pools designed on the basis of the nucleotide sequence of the β-HCG regulatory region disclosed herein. The template for the reaction may be cDNA obtained by reverse transcription of the mRNA prepared from, for example, bovine or other non-human cell lines, or tissue known to express the β-HCG gene. For guidance regarding such conditions, see, e.g., Innis et al. (Eds.) 1995, PCR Strategies, Academic Press Inc., San Diego; and Erlich (ed) 1992, PCR Technology, Oxford University Press, New York, each of which is incorporated herein by reference in its entirety.

Promoter sequences within the 5′ non-coding regions of the β-HCG gene may be further defined by constructing nested 5′ and/or 3′ deletions using conventional techniques such as exonuclease III or appropriate restriction endonuclease digestion. The resulting deletion fragments can be inserted into the promoter reporter vector to determine whether the deletion has reduced or obliterated promoter activity, such as described, for example, by Coles et al. (Hum. Mol. Genet., 7:791-800, 1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. These types of assays are well known to those skilled in the art (WO 97/17359, U.S. Pat. No. 5,374,544, EP 582 796, U.S. Pat. No. 5,698,389, U.S. Pat. No. 5,643,746, U.S. Pat. No. 5,502,176, and U.S. Pat. No. 5,266,488).

The β-HCG regulatory region, and transcriptionally functional fragments thereof, and the fragments and probes described herein which serve to identify β-HCG regulatory regions and fragments thereof, may be produced by recombinant DNA technology using techniques well known in the art. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

Alterations in the regulatory sequences can be generated using a variety of chemical and enzymatic methods which are well known to those skilled in the art. For example, regions of the sequences defined by restriction sites can be deleted. Oligonucleotide-directed mutagenesis can be employed to alter the sequence in a defined way and/or to introduce restriction sites in specific regions within the sequence. Additionally, deletion mutants can be generated using DNA nucleases such as Bal31, ExoIII, or S1 nuclease. Progressively larger deletions in the regulatory sequences are generated by incubating the DNA with nucleases for increased periods of time (see, e.g., Ausubel et al., 1989, supra).

The altered sequences are evaluated for their ability to direct expression of heterologous coding sequences in appropriate host cells. It is within the scope of the present invention that any altered regulatory sequences which retain their ability to direct expression of a coding sequence be incorporated into recombinant expression vectors for further use.

### 5.2 Analysis of Tumor-Specific Promoter Activity

The β-HCG gene regulatory region shows selective tissue and cell-type specificity; i.e., it induces gene expression in primary, metastatic and recurrent testicular carcinomas. Thus, the regulatory region, and transcriptionally active fragments thereof, of the present invention may be used to induce expression of a heterologous gene in tumor cells. The present invention relates to the use of the β-HCG gene regulatory region to achieve tissue specific expression of a target gene. The activity and the specificity of the β-HCG regulatory region can further be assessed by monitoring the expression level of a detectable polynucleotide operably associated with the β-HCG promoter in different types of cells and tissues. As discussed hereinbelow, the detectable polynucleotide may be either a polynucleotide that specifically hybridizes with a predefined oligonucleotide probe, or a polynucleotide encoding a detectable protein.

### 5.2.1 β-HCG Promoter Driven Reporter Constructs

The regulatory polynucleotides according to the invention may be advantageously part of a recombinant expression vector that may be used to express a coding sequence, or reporter gene, in a desired host cell or host organism. The β-HCG regulatory region of the present invention, and transcriptionally active fragments thereof, may be used to direct the expression of a heterologous coding sequence. In accordance with the present invention, transcriptionally active fragments of the β-HCG regulatory region encompass those fragments of the region which are of sufficient length to promote transcription of a reporter coding sequence to which the fragment is operatively linked.

A variety of reporter gene sequences well known to those of skill in the art can be utilized, including, but not limited to, genes encoding fluorescent proteins such as green fluorescent protein (GFP), enzymes (e.g. CAT, beta-galactosidase, luciferase) or antigenic markers. For convenience, enzymatic reporters and light-emitting reporters analyzed by colorometric or fluorometric assays are preferred for the screening assays of the invention.

In one embodiment, for example, a bioluminescent, chemiluminescent or fluorescent protein can be used as a light-emitting reporter in the invention. Types of light-emitting reporters, which do not require substrates or cofactors, include, but are not limited to the wild-type green fluorescent protein (GFP) of Victoria aequoria (Chalfie et al., 1994, Science 263:802-805), and modified GFPs (Heim et al., 1995, Nature 373:663-4; PCT publication WO 96/23810). Transcription and translation of this type of reporter gene leads to the accumulation of the fluorescent protein in test cells, which can be measured by a fluorimeter, or a flow cytometer, for example, by methods that are well known in the art (see, e.g., Lackowicz, 1983, Principles of Fluorescence Spectroscopy, Plenum Press, New York).

Another type of reporter gene that may be used are enzymes that require cofactor(s) to emit light, including, but not limited to, Renilla luciferase. Other sources of luciferase also are well known in the art, including, but not limited to, the bacterial luciferase (luxAB gene product) of Vibrio harveyi (Karp, 1989, Biochim. Biophys. Acta 1007:84-90; Stewart et al. 1992, J. Gen. Microbiol, 138:1289-1300), and the luciferase from firefly, Photinus pyralis (De Wet et al. 1987, Mol. Cell. Biol. 7:725-737), which can be assayed by light production (Miyamoto et al., 1987, J. Bacteriol. 169:247-253; Loessner et al. 1996, Environ. Microbiol. 62:1133-1140; and Schultz & Yarus, 1990, J. Bacteriol. 172:595-602).

Reporter genes that can be analyzed using colorimetric analysis include, but are not limited to, β-galactosidase (Nolan et al. 1988, Proc. Natl. Acad. Sci. USA 85:2603-07), β-glucuronidase (Roberts et al. 1989, Curr. Genet. 15:177-180), luciferase (Miyamoto et al., 1987, J. Bacteriol. 169:247-253), or β-lactamase. In one embodiment, the reporter gene sequence comprises a nucleotide sequence which encodes a LacZ gene product, β-galactosidase. The enzyme is very stable and has a broad specificity so as to allow the use of different histochemical, chromogenic or fluorogenic substrates, such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988, supra).

In another embodiment, the product of the E. coli β-glucuronidase gene (GUS) can be used as a reporter gene (Roberts et al. 1989, Curr. Genet. 15:177-180). GUS activity can be detected by various histochemical and fluorogenic substrates, such as X-glucuronide (Xgluc) and 4-methylumbelliferyl glucuronide.

In addition to reporter gene sequences such as those described above, which provide convenient colorimetric responses, other reporter gene sequences, such as, for example, selectable reporter gene sequences, can routinely be employed. For example, the coding sequence for chloramphenicol acetyl transferase (CAT) can be utilized, leading to β-HCG regulatory region-dependent expression of chloramphenicol resistant cell growth. The use of CAT and the advantages of a selectable reporter gene are well known to those skilled in the art (Eikmanns et al. 1991, Gene 102:93-98). Other selectable reporter gene sequences also can be utilized and include, but are not limited to, gene sequences encoding polypeptides which confer zeocin (Hegedus et al. 1998, Gene 207:241-249) or kanamycin resistance (Friedrich & Soriano, 1991, Genes. Dev. 5:1513-1523).

Other genes, such as toxic gene products, potentially toxic gene products, and antiproliferation or cytostatic gene products, also can be used. Examples of such gene products include α-fetal protein to target hepatoma cells (Kuriyama, S., et al., Cell Struct Funct, 16:503, 1991), the carcinomembryonic antigen (CEA) promoter for gastric carcinoma (Tanaka, T. et al., Cancer Res, 56:1341, 1996), the tyrosinase promoter to kill melanoma cells (Vile, R. G. et al., Cancer Res, 54:6228, 1994), the bone morphogenic protein for brain to target glial cells (Shimizu, K., Nippon Rinsho, 52: 3053, 1994) and the osteocalcin promoter to kill osteosarcoma and prostate cancer (Ko, S. C. et al., Cancer Res, 56:4614, 1996; Gardner, T. A. et al., Gene Therapy and Molecular Biology, 2:41, 1998). In another embodiment, the detectable reporter polynucleotide may be either a polynucleotide that specifically hybridizes with a predefined oligonucleotide probe, or a polynucleotide encoding a detectable protein, including an β-HCG polypeptide or a fragment or a variant thereof. This type of assay is well known to those skilled in the art (U.S. Pat. No. 5,502,176 and U.S. Pat. No. 5,266,488).

β-HCG driven reporter constructs can be constructed according to standard recombinant DNA techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York, each of which is incorporated herein by reference in its entirety).

Methods for assaying promoter activity are well-known to those skilled in the art (see, e.g., Sambrook et al., Molecular Cloning A Laboratory Manual, Cold Spring Harbor Laboratory, Cold Spring Harbor, N.Y., 1989). An example of a typical method that can be used involves a recombinant vector carrying a reporter gene and genomic sequences from β-HCG genomic sequence. Briefly, the expression of the reporter gene (for example, green fluorescent protein, luciferase, β-galactosidase or chloramphenicol acetyl transferase) is detected when placed under the control of a biologically active polynucleotide fragment. Genomic sequences located upstream of the first exon of the gene may be cloned into any suitable promoter reporter vector. For example, a number of commercially available vectors can be engineered to insert the β-HCG regulatory region of the invention for expression in mammalian host cells. Non-limiting examples of such vectors are pSEAPBasic, pSEAP-Enhancer, pβgal-Basic, pβgal-Enhancer, or pEGFP-1 Promoter Reporter vectors (Clontech, Palo Alto, Calif.) or pGL2-basic or pGL3-basic promoterless luciferase reporter gene vector (Promega, Madison, Wis.). Each of these promoter reporter vectors include multiple cloning sites positioned upstream of a reporter gene encoding a readily assayable protein such as secreted alkaline phosphatase, green fluorescent protein, luciferase or β-galactosidase. The regulatory sequences of the β-HCG gene are inserted into the cloning sites upstream of the reporter gene in both orientations and introduced into an appropriate host cell. The level of reporter protein is assayed and compared to the level obtained with a vector lacking an insert in the cloning site. The presence of an elevated expression level in the vector containing the insert with respect the control vector indicates the presence of a promoter in the insert.

Expression vectors that comprise a β-HCG gene regulatory region may further contain a gene encoding a selectable marker. A number of selection systems may be used, including but not limited to, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026) and adenine phosphoribosyltransferase (Lowy et al., 1980, Cell 22:817) genes, which can be employed in tk⁻, hgprt⁻or aprt⁻cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10:169).

### 5.2.2 Characterization of Transcriptionally Active Regulatory Fragments

A fusion construct comprising a β-HCG regulatory region, or a fragment thereof, can be assayed for transcriptional activity. As a first step in promoter analysis, the transcriptional start point (+1 site) of the tumor-specific gene under study has to be determined using primer extension assay and/or RNAase protection assay, following standard methods (Sambrook et al., 1989, Molecular Cloning: A Laboratory Manual, Cold Spring Harbor, Cold Spring Harbor Press). The DNA sequence upstream of the +1 site is generally considered as the promoter region responsible for gene regulation. However, downstream sequences, including sequences within introns, also may be involved in gene regulation. To begin testing for promoter activity, a −3 kb to +3 kb region (where +1 is the transcriptional start point) may be cloned upstream of the reporter gene coding region. Two or more additional reporter gene constructs also may be made which contain 5′ and/or 3′ truncated versions of the regulatory region to aid in identification of the region responsible for tumor-specific expression. The choice of the type of reporter gene is made based on the application.

In a preferred embodiment, a GFP reporter gene construct is used. The application of green fluorescent protein (GFP) as a reporter is particularly useful in the study of tumor-specific gene promoters. A major advantage of using GFP as a reporter lies in the fact that GFP can be detected in freshly isolated tumor without the need for substrates. In another embodiment of the invention, a luciferase reporter construct is used.

For promoter analysis in transgenic mice, GFP that has been optimized for expression in mammalian cells is preferred. The promoterless cloning vector pEGFP1 (Clontech, Palo Alto, Calif.) encodes a red shifted variant of the wild-type GFP which has been optimized for brighter fluorescence and higher expression in mammalian cells (Cormack et al., 1996, Gene 173:33; Haas et al., 1996, Curr. Biol. 6: 315). Moreover, since the maximal excitation peak of this enhanced GFP (EGFP) is at 488 nm, commonly used filter sets such as fluorescein isothiocyanate (FITC) optics which illuminate at 450-500 nm can be used to visualize GFP fluorescence. pEGFP1 proved to be useful as a reporter vector for promoter analysis in transgenic mice (Okabe et al, 1997, FEBS Lett. 407: 313). In an alternate embodiment, transgenic mice containing transgenes with a β-HCG regulatory region upstream of the luciferase reporter gene are utilized.

Putative promoter fragments can be prepared (usually from a parent phage clone containing 8-10 kb genomic DNA including the promoter region) for cloning using methods known in the art. In one embodiment, for example, promoter fragments are cloned into the multiple cloning site of a luciferase reporter vector. In one embodiment, restriction endonucleases are used to excise the regulatory region fragments to be inserted into the reporter vector. However, the feasibility of this method depends on the availability of proper restriction endonuclease sites in the regulatory fragment. In a preferred embodiment, the required promoter fragment is amplified by polymerase chain reaction (PCR; Saiki et al., 1988, Science 239:487) using oligonucleotide primers bearing the appropriate sites for restriction endonuclease cleavage. The sequence necessary for restriction cleavage is included at the 5′ end of the forward and reverse primers which flank the regulatory fragment to be amplified. After PCR amplification, the appropriate ends are generated by restriction digestion of the PCR product. The promoter fragments, generated by either method, are then ligated into the multiple cloning site of the reporter vector following standard cloning procedures (Sambrook et al., 1989, supra). It is recommended that the DNA sequence of the PCR generated promoter fragments in the constructs be verified prior to generation of transgenic animals. The resulting reporter gene construct will contain the putative promoter fragment located upstream of the reporter gene open reading frame, e.g., GFP or luciferase cDNA.

In a preferred embodiment, the following protocol is used. Fifty to 100 pg of the reporter gene construct is digested using appropriate restriction endonucleases to release the transgene fragment. The restriction endonuclease cleaved products are resolved in a 1% (w/v) agarose gel containing 0.5 ug/ml ethidium bromide and TAE buffer (1×: 0.04 M Tri-acetate, 0.001 M EDTA, pH 8.0) at 5-6 V/cm. The transgene band is located by size using a UV transilluminator, preferably using long-wavelength UV lamp to reduce nicking of DNA, and the gel piece containing the required band carefully excised. The gel slice and 1 ml of 0.5×TAE buffer is added to a dialysis bag, which has been boiled in 1 mM EDTA, pH 8.0 for 10 minutes (Sambrook et al., 1989, supra) and the ends are fastened. The dialysis bag containing the gel piece is submerged in a horizontal gel electrophoresis chamber containing 0.5×TAE buffer, and electrophoresed at 5-6 V/cm for 45 minutes. The current flow in the electrophoresis chamber is reversed for one minute before stopping the run to release the DNA which may be attached to the wall of the dialysis tube. The TAE buffer containing the electroeluted DNA from the dialysis bag is collected in a fresh eppendorf tube. The gel piece may be observed on the UV transilluminator to ascertain that the electroelution of the DNA is complete.

The electroeluted DNA sample is further purified by passing through Elutip D columns. The matrix of the column is prewashed with 1-2 ml of High salt, buffer (1.0 M NaCl, 20 mM Tris. Cl, 1.0 mM EDTA, pH 7.5), followed by a wash with 5 ml of low salt buffer (0.2 M NaCl, 20 mM Tris. Cl, 1.0 mM EDTA, pH 7.5). A 5 ml syringe is used to apply solutions to the Elutip D column, avoiding reverse flow. The solution containing the electroeluted DNA is loaded slowly. The column is washed with 2-3 ml of low salt buffer and the DNA is eluted in 0.4 ml of high salt buffer. Two volumes of cold 95% ethanol is added to precipitate DNA. The DNA is collected by centrifugation in a microcentrifuge at 14,000×g for 10 minutes, carefully removing the alcohol without disrupting the DNA pellet. The pellet is washed at least twice with 70% (v/v) ethanol, and dried. The washing and drying steps are important, as residual salt and ethanol are lethal to the developing embryos. The DNA is resuspend in the injection buffer (10 mM TM, 0.1 mM EDTA, pH 7.5 prepared with Milli-Q quality water). The concentration of the purified transgene DNA fragment is determined by measuring the optical density at A₂₆₀ (A₂₆₀=1 for 50 μg/ml DNA) using a spectrophotometer. DNA prepared in this manner is suitable for microinjection into fertilized mouse eggs.

### 5.2.3 Tumor-Specific Promoter Analysis Using Transgenic Mice

The β-HCG regulatory region can be used to direct expression of, inter alia, a reporter coding sequence, a homologous gene or a heterologous gene in transgenic animals. Animals of any species, including, but not limited to, mice, rats, rabbits, guinea pigs, pigs, micro-pigs, goats, sheep, and non-human primates, e.g., baboons, monkeys and chimpanzees may be used to generate transgenic animals. The term “transgenic,” as used herein, refers to animals expressing β-HCG gene sequences from a different species (e.g., mice expressing β-HCG sequences), as well as animals that have been genetically engineered to over-express endogenous (i.e., same species) β-HCG sequences or animals that have been genetically engineered to knock-out specific sequences.

In one embodiment, the present invention provides for transgenic animals that carry a transgene such as a reporter gene, therapeutic and/or toxic coding sequence under the control of the β-HCG regulatory region, or transcriptionally active fragments thereof, in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). When it is desired that the transgene be integrated into the chromosomal site of the endogenous corresponding gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene.

Any technique known in the art may be used to introduce a transgene under the control of the β-HCG regulatory region into animals to produce the founder lines of transgenic animals. Such techniques include, but are not limited to, pronuclear microinjection (Hoppe & Wagner, 1989, U.S. Pat. No. 4,873,191); nuclear transfer into enucleated oocytes of nuclei from cultured embryonic, fetal or adult cells induced to quiescence (Campbell et al., 1996, Nature 380:64-66; Wilmut et al., Nature 385:810-813); retrovirus gene transfer into germ lines (Van der Putten et al., 1985, Proc. Natl. Acad. Sci., USA 82:6148-6152); gene targeting in embryonic stem cells (Thompson et al., 1989, Cell 65:313-321); electroporation of embryos (Lo, 1983, Mol. Cell. Biol. 31:1803-1814); and sperm-mediated gene transfer (Lavitrano et al., 1989, Cell 57:717-723; see, Gordon, 1989, Transgenic Animals, Intl. Rev. Cytol. 115:171-229).

For example, for microinjection of fertilized eggs, a linear DNA fragment (the transgene) containing the regulatory region, the reporter gene and the polyadenylation signals, is excised from the reporter gene construct. The transgene may be gel purified by methods known in the art, for example, by the electroelution method. Following electroelution of gel fragments, any traces of impurities are further removed by passing through Elutip D column (Schleicher & Schuell, Dassel, Germany).

In a preferred embodiment, the purified transgene fragment is microinjected into the male pronuclei of fertilized eggs obtained from B6 CBA females by standard methods (Hogan, 1986, Manipulating the Mouse Embryo, A Laboratory Manual. Cold Spring Harbor Laboratory Press, Cold Spring Harbor, N.Y.). Mice are analyzed transiently at several embryonic stages or by establishing founder lines that allow more detailed analysis of transgene expression throughout development and in adult animals. Transgene presence is analyzed by PCR using genomic DNA purified from placentas (transients) or tail clips (founders) according to the method of Vemet et al., Methods Enzymol. 1993; 225:434-451. Preferably, the PCR reaction is carried out in a volume of 100 μl containing 1 μg of genomic DNA, in 1× reaction buffer supplemented with 0.2 mM dNTPs, 2 mM MgCl₂, 600 μM each of primer, and 2.5 units of Taq polymerase (Promega, Madison, Wis.). Each of the PCR cycles consists of denaturation at 94° C. for 1 min, annealing at 54° C. for 1 min, and extension at 72° C. for 1 min. The founder mice are then mated with C57B1 partners to generate transgenic F₁ lines of mice.

### 5.3 Screening Assays

Compounds that interfere the tumorigenesis and/or the progression of cancer can provide therapies targeting defects in various cancers. Such compounds may be used to interfere with the onset or the progression of the various cancers. Compounds that stimulate or inhibit promoter activity also may be used to ameliorate symptoms of the cancers.

Genetically engineered cells, cell lines, cancer cells, and/or transgenic animals containing a β-HCG regulatory region, or fragment thereof, operably linked to a reporter gene, can be used as systems for the screening of agents that modulate β-HCG transcriptional activity. In addition, β-HCG containing transgenic mice may provide an experimental model both in vivo and in vitro to develop new methods of treating various cancers, including, but not limited to, primary, metastatic and recurrent testicular carcinomas by targeting drugs to cause arrest in the progression of such disorders.

The present invention encompasses screening assays designed to identify compounds that modulate activity of the β-HCG regulatory region. The present invention encompasses in vitro and cell-based assays, as well as in vivo assays in transgenic animals. As described hereinbelow, compounds to be tested may include, but are not limited to, oligonucleotides, peptides, proteins, small organic or inorganic compounds, antibodies, etc.

Examples of compounds may include, but are not limited to, peptides, such as, for example, soluble peptides, including, but not limited to, Ig-tailed fusion peptides, and members of random peptide libraries; (see, e.g., Lam, et al., 1991, Nature 354:82-84; Houghten, et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Such compounds may further comprise compounds, in particular drugs or members of classes or families of drugs, known to ameliorate the symptoms of various cancers.

Such compounds include, but are not limited to, families of antidepressants such as lithium salts, carbamazepine, valproic acid, lysergic acid diethylamide (LSD), p-chlorophenylalanine, p-propyldopacetamide dithiocarbamate derivatives e.g., FLA 63; antianxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, α-methyl-tyrosine, reserpine, tetrabenazine, benzotropine, pargyline; noradrenergic agonists and antagonists e.g., clonidine, phenoxybenzamine, phentolamine, tropolone; nitrovasodilators (e.g., nitroglycerine, nitroprusside as well as NO synthase enzymes); and growth factors (e.g., VEGF, FGF, angiopoetins and endostatin).

In one preferred embodiment, genetically engineered cells, cells lines or primary cultures of germ cells and/or somatic cells containing a β-HCG regulatory region operatively linked to a heterologous gene are used to develop assay systems to screen for compounds which can inhibit sequence-specific DNA-protein interactions. Such methods comprise contacting a compound to a cell that expresses a gene under the control of an β-HCG regulatory region, or a transcriptionally active fragment thereof, measuring the level of the gene expression or gene product activity and comparing this level to the level of gene expression or gene product activity produced by the cell in the absence of the compound, such that if the level obtained in the presence of the compound differs from that obtained in its absence, a compound capable of modulating the expression of the β-HCG regulatory region has been identified. Alterations in gene expression levels may be by any number of methods known to those of skill in the art e.g., by assaying for reporter gene activity, assaying cell lysates for mRNA transcripts, e.g. by Northern analysis or using other methods known in the art for assaying for gene products expressed by the cell.

In another embodiment, microdissection and transillumination can be used. These techniques offer a rapid assay for monitoring effects of putative drugs on tumor cells in transgenic animals containing an β-HCG regulatory region-driven reporter gene. In this embodiment, a test agent is delivered to the transgenic animal by any of a variety of methods. Methods of introducing a test agent may include oral, intradermal, intramuscular, intraperitoneal, intravenous, subcutaneous, intranasal and via scarification (scratching through the top layers of skin, e.g., using a bifurcated needle) or any other standard routes of drug delivery. The effect of such test compounds on the tumor cell can be analyzed by the microdissection and transillumination of the tumor cell. If the level of reporter gene expression observed or measured in the presence of the compound differs from that obtained in its absence, a compound capable of modulating the expression of the β-HCG regulatory region has been identified.

In various embodiments of the invention, compounds that may be used in screens for modulators of tumor-related disorders include peptides, small molecules, both naturally occurring and/or synthetic (e.g., libraries of small molecules or peptides), cell-bound or soluble molecules, organic, non-protein molecules and recombinant molecules that may have β-HCG regulatory region binding capacity and, therefore, may be candidates for pharmaceutical agents.

Alternatively, the proteins and compounds include endogenous cellular components which interact with β-HCG regulatory region sequences in vivo. Cell lysates or tissue homogenates may be screened for proteins or other compounds which bind to the β-HCG regulatory region, or fragment thereof. Such endogenous components may provide new targets for pharmaceutical and therapeutic interventions.

In one embodiment, libraries can be screened. Many libraries are known in the art that can be used, e.g., peptide libraries, chemically synthesized libraries, recombinant (e.g., phage display libraries), and in vitro translation-based libraries. In one embodiment of the present invention, peptide libraries may be used to screen for agonists or antagonists of β-HCG-linked reporter expression. Diversity libraries, such as random or combinatorial peptide or non-peptide libraries can be screened for molecules that specifically modulate β-HCG regulatory region activity. Random peptide libraries consisting of all possible combinations of amino acids attached to a solid phase support may be used to identify peptides that are able to activate or inhibit β-HCG regulatory region activities (Lam, K. S. et al., 1991, Nature 354: 82-84). The screening of peptide libraries may have therapeutic value in the discovery of pharmaceutical agents that stimulate or inhibit the expression of β-HCG by interaction with the promoter region.

Examples of chemically synthesized libraries are described in Fodor et al., 1991, Science 251:767-773; Houghten et al., 1991, Nature 354:84-86; Lam et al., 1991, Nature 354:82-84; Medynski, 1994, BioTechnology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of example of non-peptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) also can be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

A specific embodiment of such an in vitro screening assay is described below. The β-HCG regulatory region-reporter vector is used to generate transgenic mice from which primary cultures of β-HCG regulatory region-reporter vector germ cells are established. About 10,000 cells per well are plated in 96-well plates in total volume of 100 μl, using medium appropriate for the cell line. Candidate inhibitors of β-HCG gene expression are added to the cells. The effect of the inhibitors of β-HCG gene activation can be determined by measuring the response of the reporter gene driven by the β-HCG regulatory region. This assay could easily be set up in a high-throughput screening mode for evaluation of compound libraries in a 96-well format that reduce (or increase) reporter gene activity, but which are not cytotoxic. After 6 hours of incubation, 100 μl DMEM medium+2.5% fetal bovine serum (FBS) to 1.25% final serum concentration is added to the cells, which are incubated for a total of 24 hours (18 hours more). At 24 hours, the plates are washed with PBS, blot dried, and frozen at −80° C. The plates are thawed the next day and analyzed for the presence of reporter activity.

In a preferred example of an in vivo screening assay, tumor cells derived from transgenic mice can be transplanted into mice with a normal or other desired phenotype (Brinster et al., 1994, Proc. Natl. Acad. Sci. USA 91: 11298-302; Ogawa et al., 1997, Int. J. Dev. Biol. 41:111-12). Such mice can then be used to test the effect of compounds and other various factors on tumor-related disorders. In addition to the compounds and agents listed above, such mice can be used to assay factors or conditions that can be difficult to test using other methods, such as dietary effects, internal pH, temperature, etc.

Once a compound has been identified that inhibits or enhances β-HCG regulatory region activity, it may then be tested in an animal-based assay to determine if the compound exhibits the ability to act as a drug to ameliorate symptoms of various cancers including, but not limited to, primary, metastatic and recurrent testicular carcinomas.

The assays of the present invention may be first optimized on a small scale (i.e., in test tubes), and then scaled up for high-throughput assays. The screening assays of the present invention may be performed in vitro, i.e., in test tubes, using purified components or cell lysates. The screening assays of the present invention may also be carried out in intact cells in culture and in animal models. In accordance with the present invention, test compounds which are shown to modulate the activity of the β-HCG regulatory region in vitro, as described herein, will further be assayed in vivo in cultured cells and animal models to determine if the test compound has the similar effects in vivo and to determine the effects of the test compound on various cancers.

### 5.4 Compositions and Methods for Therapeutic Use of MN-CA9 Regulatory Region Nucleotides

β-HCG polynucleotides, or transcriptionally active fragments thereof, can be used to treat diseases, conditions or disorders that can be ameliorated by modifying the level or the expression of β-HCG, or a heterologous gene linked to an β-HCG regulatory region, in a tumor-specific manner. Described herein are methods for such therapeutic treatments.

The β-HCG regulatory region may be used to achieve tissue specific expression in gene therapy protocols. In cases where such cells are tumor cells, the induction of a cytotoxic product by the β-HCG regulatory region may be used in the form of cancer gene therapy specifically targeted to tumor cells which contain trans-acting factors required for β-HCG expression. In this way, the β-HCG regulatory region may serve as a delivery route for a gene therapy approach to various cancers which express the β-HCG protein. Examples of these cancers include, but are not limited to, primary, metastatic and recurrent testicular carcinomas. Additionally, antisense, antigene or aptameric oligonucleotides may be delivered to cells using the presently described expression constructs. Ribozymes or single-stranded RNA also can be expressed in a cell to inhibit the expression of a target gene of interest. The target genes for these antisense or ribozyme molecules should be those encoding gene products that are essential for cell maintenance.

The β-HCG regulatory region, and transcriptionally active fragments thereof, of the present invention may be used for a wide variety of purposes, e.g., to down regulate β-HCG gene expression, or, alternatively, to achieve tumor-specific, stage-specific expression of heterologous genes.

In one embodiment, for example, the endogenous β-HCG regulatory region may be targeted to specifically down-regulate expression of the β-HCG gene. For example, oligonucleotides complementary to the regulatory region may be designed and delivered to the cells. Such oligonucleotides may anneal to the regulatory sequence and prevent transcription activation. Alternatively, the regulatory sequence, or portions thereof, may be delivered to cells in saturating concentrations to compete for transcription factor binding. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11:155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In another embodiment, a gene therapy method for ameliorating various cancers is provided. β-HCG regulatory region sequences are used to drive tumor-specific expression of drugs or toxins and introduced in the tumors. The method comprises introducing an β-HCG regulatory region sequence operatively associated with a drug or toxin gene into the tumor.

In yet another embodiment, the invention provides a gene therapy method for treatment of cancer or other proliferative disorders. The β-HCG regulatory region is used to direct the expression of one or more proteins specifically in tumor cells of a patient. Such proteins may be, for example, tumor suppressor genes, thymidine kinase (used in combination with acyclovir), toxins or proteins involved in cell killing, such as proteins involved in the apoptosis pathway.

In one embodiment, the invention provides for a therapeutic agent comprising an β-HCG promoter which is useful for toxic gene therapy. This method includes a eukaryotic delivery vector and a toxic gene. In the preferred embodiment, the vector is adenovirus (Ad) and the gene is thymidine kinase (TK). Thus, the therapeutic agent is represented by the formula Ad-β-HCG-TK, but in reality the novel concept contained herein is the β-HCG promoter as the driving force for cancer-specific expression of heterologous coding sequences.

The DNA encoding the translational or transcriptional products of interest may be engineered recombinantly into a variety of vector systems that provide for replication of the DNA in large scale for the preparation of the vectors of the invention. These vectors can be designed to contain the necessary elements for directing the transcription and/or translation of the DNA sequence taken up by the cancer cells.

Vectors that may be used include, but are not limited to, those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Vectors that allow for the in vitro transcription of RNA, such as SP6 vectors, also may be used to produce large quantities of RNA that may be incorporated into viral vectors.

Alternatively, recombinant replication competent or incompetent viral vectors including, but not limited to, those derived from viruses such as herpes virus, retroviruses, vaccinia viruses, adenoviruses, adeno-associated viruses or bovine papilloma virus may be engineered. While integrating vectors may be used, non-integrating systems, which do not transmit the gene product to daughter cells for many generations, are preferred for non-disease related repair and regeneration. In this way, the gene product is expressed during the repair process, and as the gene is diluted out in progeny generations, the amount of expressed gene product is diminished.

The use of tissue specific promoters to drive therapeutic gene expression would decrease further a toxic effect of the therapeutic gene on neighboring normal cells when virus-mediated gene delivery results in the infection of the normal cells. This would be important especially in diseases where systemic administration could be utilized to deliver a therapeutic vector throughout the body, while maintaining transgene expression to a limited and specific number of cell types. Moreover, since many bone growth factors, such as TGF-β, have pleiotropic effects, numerous, harmful side effects likely would be exhibited if the growth factor genes are expressed in all cells.

In some instances, the promoter elements may be constitutive or inducible promoters and can be used under the appropriate conditions to direct high level or regulated expression of the gene of interest. Expression of genes under the control of constitutive promoters does not require the presence of a specific substrate to induce gene expression and will occur under all conditions of cell growth. In contrast, expression of genes controlled by inducible promoters is responsive to the presence or absence of an inducing agent. For example, if a cell is stably transfected with a therapeutic, inducible transgene, its expression could be controlled over the life-time of the individual.

Specific initiation signals also are required for sufficient translation of inserted protein coding sequences. These signals include the ATG initiation codon and adjacent sequences. In cases where the entire coding sequence, including the initiation codon and adjacent sequences, are inserted into the appropriate expression vectors, no additional translational control signals may be needed. However, in cases where only a portion of the coding sequence is inserted, exogenous translational control signals, including the ATG initiation codon, must be provided. Furthermore, the initiation codon must be in phase with the reading frame of the protein coding sequences to ensure translation of the entire insert. These exogenous translational control signals and initiation codons can be of a variety of origins, both natural and synthetic. The efficiency and control of expression may be enhanced by the inclusion of transcription attenuation sequences, enhancer elements, etc.

In another embodiment of the present invention there is provided a method for treating cancers or other proliferative disorders comprising delivering a therapeutic agent to the tumor. The therapeutic agent comprises a recombinant adenovirus vector (Ad) containing an β-HCG promoter driven toxic thymidine kinase (Tk). An additional aspect of the present invention provides a method of regulating expression of Tk with the addition of a suitable prodrug including, but not limited to, acyclovir (AcV). The therapeutic agent containing the β-HCG promoter-driven toxic gene therapy, in the presence of a suitable prodrug, can be administered to cancers, including, but not limited to, primary, metastatic and recurrent testicular carcinomas.

In yet another embodiment, the β-HCG regulatory region may code for a variety of genes with immune modulatory functions, e.g. for cytokines such as interleukins 1 to 15 inclusive, especially for example IL2, IL12, gamma-interferon, tumour necrosis factor, GMCSF, and/or other genes, e.g. those mentioned in specifications WO 88/00971 (CSIRO, Australian National University: Ramshaw et al) and WO 94/16716 (Virogenetics Corp; Paoletti et al).

Also the following genes can be encoded by the β-HCG regulatory regions of the invention: genes for interferons alpha, beta or gamma; tumour necrosis factor; granulocyte-macrophage colony-stimulating factor (GM-CSF), granulocyte colony-stimulating factor (G-CSF), macrophage colony-stimulating factor (N-CSF), chemokines such as neutrophil activating protein NAP, macrophage chemoattractant and activating factor MCAF, RANTES, macrophage inflammatory peptides MIP-1a and MIP-1b, complement components and their receptors, accessory molecules such as 87.1, 87.2, ICAM-1.2 or 3 or cytokine receptors. Where nucleotide sequences encoding more than one immunomodulating protein are inserted, they may comprise more than one cytokine or may represent a combination of cytokine and accessory molecule(s).

### 5.4.1 Modulatory, Antisense, Ribozyme and Triple Helix Approaches

In another embodiment, symptoms of conditions, disorders or diseases involving tumor cells may be ameliorated by decreasing the level of β-HCG regulatory region activity by using well-known antisense, gene “knock-out,” ribozyme and/or triple helix methods to decrease the level of β-HCG regulatory region expression. Among the compounds that may exhibit the ability to modulate the activity, expression or synthesis of the β-HCG regulatory region, including the ability to ameliorate the symptoms of a various cancers and related disorders are antisense, ribozyme and triple helix molecules. Such molecules may be designed to reduce or inhibit either unimpaired, or if appropriate, mutant β-HCG regulatory region activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the gene of interest could be used in an antisense approach to inhibit translation of endogenous mRNA. Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit target gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier, et al., 1987, Nucl. Acids Res. 15:6625-6641). The oligonucleotide is a 2′-( )-methylribonucleotide (Inoue, et al., 1987, Nucl. Acids Res. 15:6131-6148), or a chimeric RNA-DNA analogue (Inoue, et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16:3209), methylphosphonate oligonucleotides can be prepared by use of controlled pore glass polymer supports (Sarin, et al., 1988, Proc. Natl. Acad. Sci. U.S.A. 85:7448-7451), etc.

While antisense nucleotides complementary to the target gene coding region sequence could be used, those complementary to the transcribed, untranslated region are most preferred.

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

A preferred approach to achieve intracellular concentrations of the antisense sufficient to suppress translation of endogenous mRNAs utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner, et al., 1981, Proc. Natl. Acad. Sci. U.S.A. 78:1441-1445), the regulatory sequences of the metallothionein gene (Brinster, et al., 1982, Nature 296:39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically via intravenous administration, oral administration or the like).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

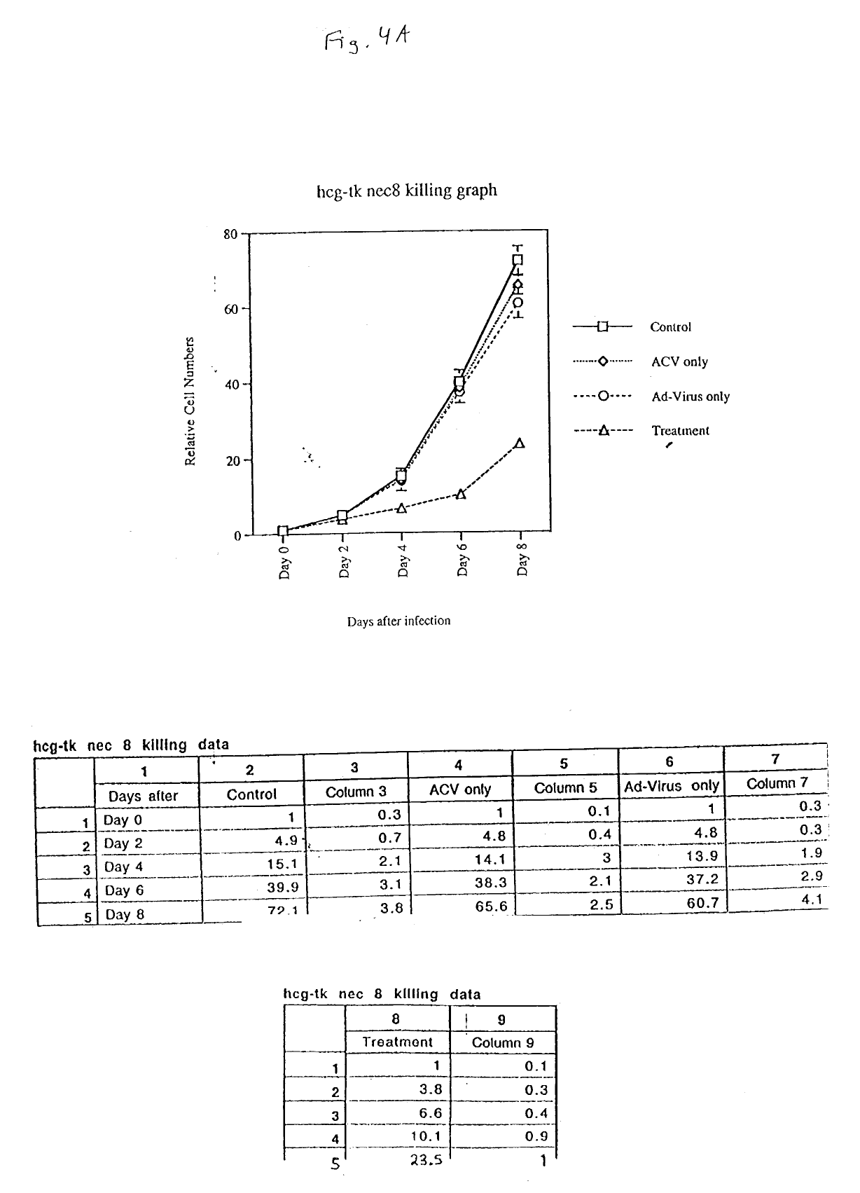

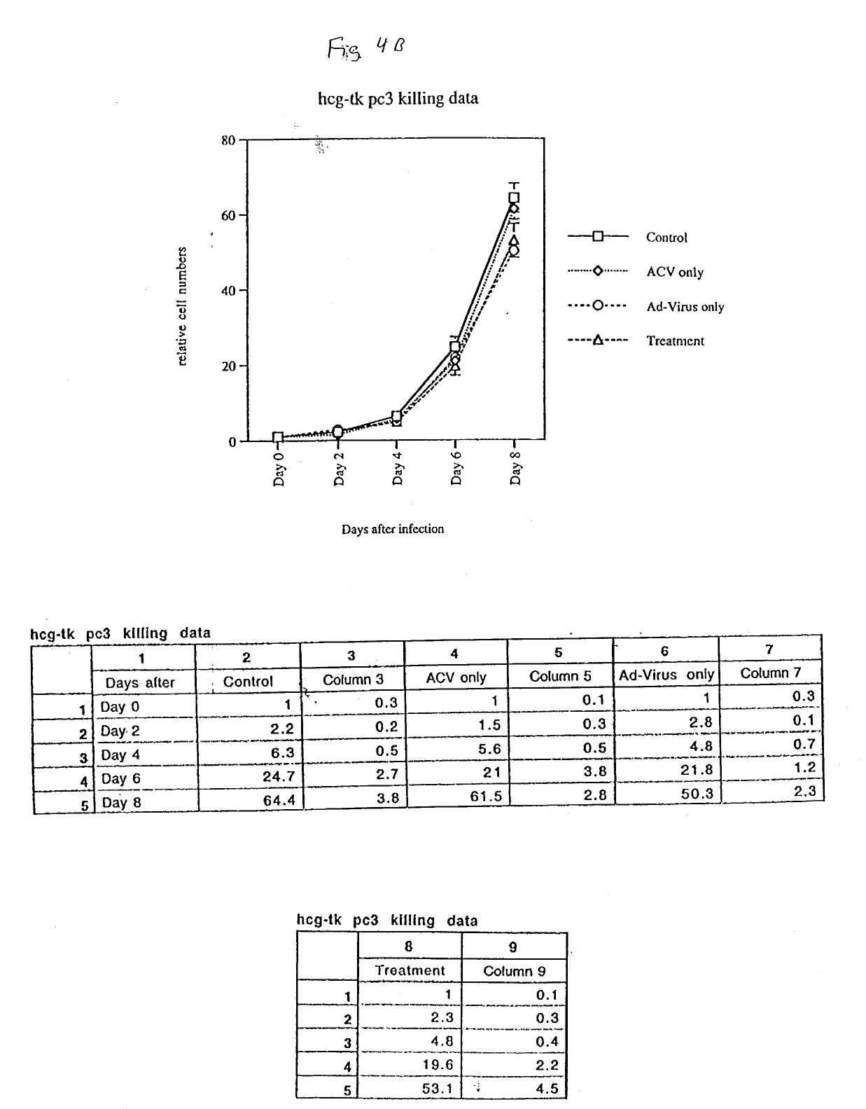

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334:585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.4.2 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### 5.4.2 Gene Replacement Therapy

The nucleic acid sequences of the invention, described above in Section 5.1, can be utilized for transferring recombinant nucleic acid sequences to cells and expressing said sequences in recipient cells. Such techniques can be used, for example, in marking cells or for the treatment of various cancers and related disorders. Such treatment can be in the form of gene replacement therapy. Specifically, one or more copies of a normal gene or a portion of the gene that directs the production of a gene product exhibiting normal gene function, may be inserted into the appropriate cells within a patient, using vectors that include, but are not limited to adenovirus, adeno-associated virus and retrovirus vectors, in addition to other particles that introduce DNA into cells, such as liposomes.

Methods for introducing genes for expression in mammalian cells are well known in the field. Generally, for such gene therapy methods, the nucleic acid is directly administered in vivo into a target cell or a transgenic mouse that expresses an β-HCG regulatory region operably linked to a heterologous coding sequencee. This can be accomplished by any method known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus or by administering it in linkage to a ligand subject to receptor-mediated endocytosis (see e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

Because the nucleic acids of the invention may be expressed in the brain, such gene replacement therapy techniques should be capable of delivering gene sequences to these cell types within patients. Thus, in one embodiment, techniques that are well known to those of skill in the art (see, e.g., PCT Publication No. WO89/10134, published Apr. 25, 1988) can be used to enable gene sequences to cross the blood-brain barrier readily and to deliver the sequences to cells in the brain. With respect to delivery that is capable of crossing the blood-brain barrier, viral vectors such as, for example, those described above, are preferable.

In another embodiment, techniques for delivery involve direct administration, e.g., by stereotactic delivery of such gene sequences to the site of the cells in which the gene sequences are to be expressed.

Additional methods that may be utilized to increase the overall level of gene expression and/or gene product activity include using targeted homologous recombination methods, as discussed above, to modify the expression characteristics of an endogenous gene in a cell or microorganism by inserting a heterologous DNA regulatory element such that the inserted regulatory element is operatively linked with the endogenous gene in question. Targeted homologous recombination can thus be used to activate transcription of an endogenous gene that is “transcriptionally silent”, i.e., is not normally expressed or is normally expressed at very low levels, or to enhance the expression of an endogenous gene that is normally expressed.

Further, the overall level of target gene expression and/or gene product activity may be increased by the introduction of appropriate target gene-expressing cells, preferably autologous cells, into a patient at positions and in numbers that are sufficient to ameliorate the symptoms of various cancers and related disorders. Such cells may be either recombinant or non-recombinant.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described above that are capable of modulating activity of a β-HCG regulatory region can be administered using standard techniques that are well known to those of skill in the art. In instances in which the compounds to be administered are to involve an interaction with brain cells, the administration techniques should include well known ones that allow for a crossing of the blood-brain barrier.

### 5.5 Pharmaceutical Preparations and Methods of Administration

The compounds that are determined to modify β-HCG regulatory region activity or gene product activity can be administered to a patient at therapeutically effective doses to treat or ameliorate various cancers and related disorders. A therapeutically effective dose refers to that amount of the compound sufficient to result in amelioration of symptoms of such a disorder.

### 5.5.1 Effective Dose

Toxicity and therapeutic efficacy of such compounds can be determined by standard pharmaceutical procedures in cell cultures or experimental animals, e.g., for determining the LD₅₀ (the-dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### 5.5.2 Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

In addition to the formulations described previously, the compounds also may be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 6. REFERENCES CITED

The invention described and claimed herein is not to be limited in scope by the specific embodiments herein disclosed since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

All publications, patents and patent applications mentioned in this specification are herein incorporated by reference to the same extent as if each individual publication, patent or patent application was specifically and individually indicated to be incorporated by reference.

What is claimed is:

1. An isolated polynucleotide comprising the nucleotide sequence depicted in FIG. 1, or a transcriptionally active fragment thereof.

2. An isolated polynucleotide that hybridizes under highly stringent conditions to the isolated polynucleotide as in claim 1, or the complement thereof.

3. A recombinant vector comprising the isolated polynucleotide of claim 2.

4. An expression vector comprising the isolated polynucleotide of claim 2 operatively associated with a regulatory nucleic acid controlling the expression of the nucleic acid in a host cell.

5. A genetically engineered cell comprising the isolated polynucleotide of Claim 2.

6. A transgenic, non-human animal, which has been genetically engineered to contain a transgene comprising the isolated polynucleotide of claim 2.

7. A therapeutic agent comprising an β-HCG promoter, a delivery vector and a toxic, therapeutic and/or heterologous coding sequence.

8. The therapeutic agent of claim 7, further comprising a prodrug.

9. The therapeutic agent of claim 8, wherein said prodrug is selected from the group consisting of acyclovir (“ACV”) and gancyclovir (“GCV”).

10. The therapeutic agent of claim 7, wherein said delivery vector comprises a viral vector.

11. The therapeutic agent of claim 10, wherein said viral vector is an adenovirus.

12. The therapeutic agent of claim 7, wherein said delivery vector comprises a liposome.

13. The therapeutic agent of claim 7, wherein said toxic coding sequence is selected from the group consisting of thymidine kinase and cytosine deaminase.

14. The therapeutic agent of claim 7, wherein said therapeutic coding sequence is selected from the group consisting of growth factors, cytokines, therapeutic proteins, hormones and peptide fragments of hormones, inhibitors of cytokines, peptide growth and differentiation factors, interleukins, chemokines, interferons, colony stimulating factors and angiogenic factors.

15. The therapeutic agent of claim 7, wherein said heterologous coding sequence is a reporter gene.

16. The therapeutic agent of claim 15, wherein said reporter gene is a luciferase.

17. A method for identifying a test compound capable of modulating tumor-specific gene expression comprising:
(a) contacting a compound to a cell that expresses a reporter gene under the control of an β-HCG regulatory region or a transcriptionally active fragment thereof; and
(b) measuring the level of the reporter gene expression in the presence and absence of said test compound,
such that if the level obtained in the presence of the test compound differs from that obtained in its absence, then a compound which modulates tumor-specific gene expression is identified.

18. The method of claim 17 wherein the reporter gene expression produces a fluorescent signal.

19. A pharmaceutical composition comprising the test compound identified in claim 17.

20. A method for drug delivery comprising introducing into a tumor of a subject a vector comprising an β-HCG regulatory region sequence, or transcriptionally active fragment thereof, operatively linked to a heterologous gene.

21. A method for treating and/or ameliorating a cancer or other proliferative disorder comprising introducing into a cell of said cancer or other proliferative disorder of a subject a vector comprising an β-HCG regulatory region sequence, or transcriptionally active fragment thereof, a delivery vector and a toxic, therapeutic and/or heterologous coding sequence whose gene product is capable of killing said cell.

22. The method of claim 21 wherein said cancer or other proliferative disorder is selected from the group consisting of primary, metastatic and recurrent testicular carcinomas.

23. The method of claim 21 further comprising introducing a prodrug.

24. The method of claim 23 wherein said prodrug is selected from the group consisting of ACV and GCV.

25. The method of claim 21 wherein said introducing comprises administration via direct application, or systemic application via intravenous administration, intra-arterial administration, intra-tumoral administration, perfusion and oral administration.
